(19) United States
(12) Patent Application Publication
(10) Pub. No.: US 2015/0021823 A1
(43) Pub. Date: Jan. 22, 2015

(54) METHOD AND APPARATUS FOR IMPARTING A CATHETER TIP TO MULTI-LAYERED TUBING

(71) Applicant: Hollister Incorporated, Libertyville, IL (US)

(72) Inventor: William L. Givens, JR., Antioch, IL (US)

(21) Appl. No.: 14/379,123

(22) PCT Filed: Feb. 21, 2013
(86) PCT No.: PCT/US13/27088

§ 371 Date: Aug. 15, 2014

Related U.S. Application Data
(60) Provisional application No. 61/602,307, filed on Feb. 23, 2012.

(51) Int. Cl. A61M 25/00

(52) U.S. Cl. CPC A61M 25/001
USPC 264/296; 425/392

(57) ABSTRACT

A method and apparatus for imparting a rounded tip to a length of multi-layered tubing to form a urinary catheter includes a plurality of dies with internal geometry to successively push back an end region of an inner layer of the multi-layered tubing, exposing an interior of an end region of an external layer, and forming that end region of the external layer into a rounded end that is free of bald spots. The end region of the external layer may be thinned out by an intermediate die having an elongate conical die plug therein. Gravity and vacuum pressure may be used to facilitate flow of material of which at least one of the layers of the multi-layered tubing is constructed when heated to a deformable condition.

### FIELD OF THE DISCLOSURE

This disclosure relates generally to the manufacture of medical devices such as catheters and, more specifically, to processes and apparatus for imparting a rounded catheter tip to a length of multi-layered tubing during the manufacture of an intermittent urinary catheter.

### BACKGROUND

Due to byproducts released upon incineration of products made of poly-vinyl chloride (PVC), many catheter manufacturers are turning away from PVC's in favor of other materials. However, PVC-free materials present challenges when tipping a length of multi-layered tubing to form a catheter. For instance, PEBAX® is a material used as the outer layer of tubing by some manufacturers of PVC-free catheters. A benefit of PEBAX® is its affinity for hydrophilic coatings. It has been found when forming tips on multi-layered PVC-free tubing that the inner layer, which typically has a lower melting point than the outer PEBAX® layer, tends to melt out and cover part of the tip of the catheter. In so covering part of the catheter tip, the ability for hydrophilic coatings to bind to the catheter tip (in order to improve lubricity during catheter insertion into the urethra) is diminished. As a result, catheters across a given manufactured lot are susceptible to being produced with regions of little or no hydrophilic coating, known in the art as “bald spots”. Therefore, a manufacturing method and apparatus that could reliably provide a catheter tip on a length of multi-layered, non-PVC tubing, without producing bald spots, would be advantageous.

### SUMMARY OF THE DISCLOSURE

A series of die stations are employed to make successive modifications to the shape of the tip of a length of multi-layered tubing. In a first station, a first die is provided with an internal geometry that serves, when applied to an exposed end region of a length of multi-layered tubing under concentrated heat, to push most of a center core of the tubing back, thereby thinning out an outer wall of the end region tubing. In a second station, a second die is provided with an internal geometry that serves, when applied to the end region of the tubing under concentrated heat, to taper the remaining outer wall thickness of the end region of the tubing to a pointed edge. In a third station, a third die is provided with an internal geometry that serves, when applied to the end region of the tubing under concentrated heat, to wrap the pointed edge (imparted to the tubing at the second station) around radially inwardly toward an axis of the tubing, thereby forming a rounded (which may include, by way of example, dome-shaped or bullet-nosed) catheter tip on the end region of the tubing. While it is disclosed that each of the plurality of dies employed in successively manipulating the end region of the tubing is provided in a separate die station, it is recognized that the plurality of dies may be provided at a single die station, such as in an interchangeable or multi-die mount, with the desired one of the plurality of dies being fixed in an active position prior to insertion of the tubing into the die station.

### BRIEF DESCRIPTION OF THE SEVERAL VIEWS OF THE DRAWING

FIG. 1 is a perspective view, partially cut-away, of a length of hollow tubing appropriate for use to form a urinary catheter;

FIG. 2 is a perspective view of one of the plurality of die stations of the present disclosure employed in imparting a rounded catheter tip to the length of tubing of FIG. 1;

FIG. 3 is a plan view of a die of a first die station of the present disclosure;

FIG. 4 is a plan view of the length of tubing of FIG. 1 after being withdrawn from the first die station of FIG. 3;

FIG. 5 is a plan view of a die of a second die station of the present disclosure;

FIG. 6 is a plan view of the length of tubing of FIG. 1 after being withdrawn from the second die station of FIG. 5;

FIG. 7 is a plan view of a die of a third die station of the present disclosure; and

FIG. 8 is a plan view of the length of tubing of FIG. 1 after being withdrawn from the third die station of FIG. 7.

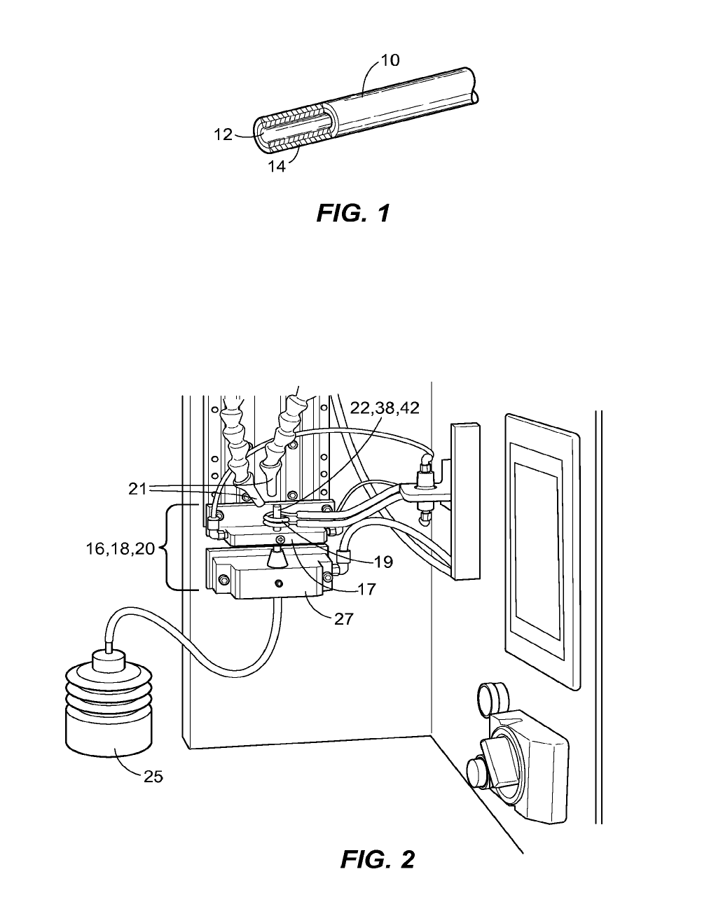

### DETAILED DESCRIPTION OF THE PREFERRED EMBODIMENTS

A length of multi-layered tubing 10 appropriate to form a urinary catheter may be made of a variety of materials. There is increased demand for PVC-free urinary catheters.

To be suitable for use as a urinary catheter, an inner or core layer 12, preferably of a non-PVC material, is typically provided with an external layer 14 of a water-swellable material, for example, water-swellable materials such as polyether/polyamide block thermoplastic elastomers sold under the tradename PEBAX® (Arkema, Pa.) and polyester elastomers such as those sold under the HYTREL® trade name (Du Pont de Nemours, Del.). The water swellable material of the external layer 14 of such multi-layered tubing are contacted with a solution to swell the water swellable material, said solution comprising at least one of (i) a water-soluble polymer capable of being cross-linked to form a cross-linked, lubricious, hydrophilic coating and (ii) a water-soluble monomer capable of being polymerized to form a cross-linked, lubricious, hydrophilic coating. The solution is typically aqueous but may also comprise alcohols, particularly lower alcohols such as methanol, ethanol, propanol, butanol, and the like. After sufficient contact time the water-swellable material is in the swollen state and becomes physically entangled with the water-soluble polymer and/or the water-soluble monomer such that those entanglements can be locked in during cross-linking of the water-soluble polymer and/or polymerization of the monomer to form a cross-linked network in situ, thereby securely anchoring the hydrophilic coating to the substrate surface. A suitable method of applying a hydrophilic coating to a layer of multi-layered tubing employed in the processes and used in the assemblies disclosed herein is that of U.S. Pat. No. No. 8,053,030, assigned to Hollister Incorporated, which is incorporated herein by reference.

The inventors have successfully imparted various multi-layered tubing compositions with a rounded tip using a method and apparatus in accordance with the teachings of the present disclosure. A first such multi-layered tubing composition included a 15.5 inch length of tubing having an outside diameter of 0.181 inch ±0.005 inch, an inside diameter of 0.122 inch ±0.005 inch, and an initial overall wall thickness of 0.030 inch made up of an outer layer wall (external layer used above) having a thickness of 0.002 inch ±0.0005 inch, of a precompounded Nucrel 2806/PEBAX® 1074 Blend, a tie layer of Nucrel 0609 AS having a thickness of 0.001 inch, and an inner layer of Exact 8210, the thickness of the inner layer making up the balance of the 0.030 inch overall wall thickness.

A second such multi-layered tubing composition included a 15.5 inch length of tubing having an outside diameter of 0.0787 inch ±0.005 inch, an inside diameter of 0.0472 inch ±0.005 inch, and an initial overall wall thickness of 0.016 inch, made up of an outer layer wall (external layer used above) having a thickness of 0.002 inch, ±0.0005 inch, of a precompounded Nucrel 2806/PEBAX ® 1074 Blend, a tie layer of Nucrel 0609 AS having a thickness of 0.001 inch, and an inner layer of Exact 8210, the thickness of the inner layer making up the balance of the 0.016 inch overall wall thickness.

A problem with conventional catheter tipping processes for PEBAX®-coated tubing is that the inner PVC-free layer tends to melt out and cover the PEBAX® coating, resulting in bald spots where a hydrophilic coating does not adhere. The method and apparatus of the present disclosure includes a plurality of die stations 16, 18, 20 that successively form an end region of the multi-layered tubing 10 into a rounded catheter tip.

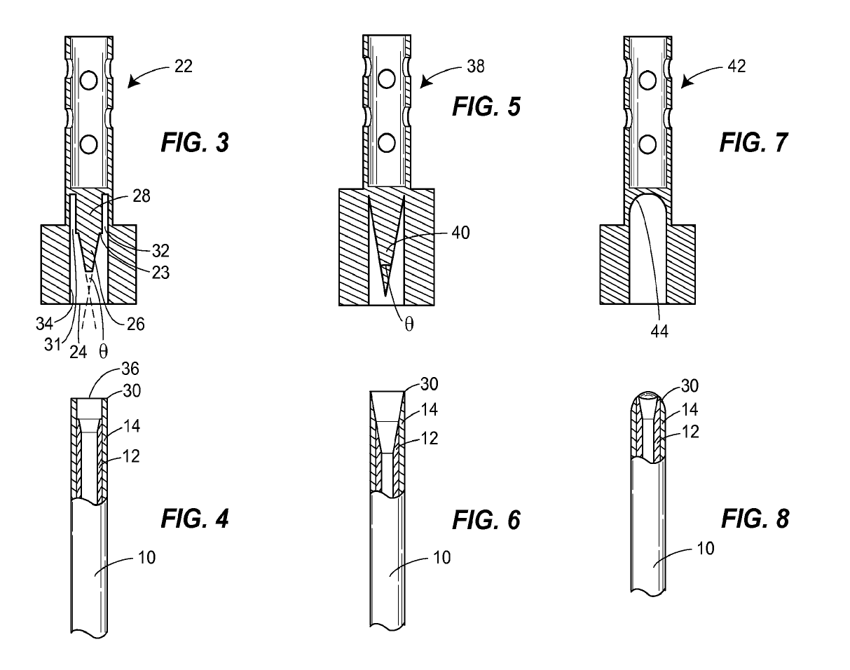

With reference to FIGS. 2-3, a first die station 16 is illustrated that includes a first die 22 provided with an aperture 24 at a lower end thereof to receive the multi-layered tubing 10. The first die station 16 includes a die mounting bracket 17, a heating element 19 (such as a heating coil), and one or more (preferably adjustable) cooling air blowers 21. Within the aperture 24 of the first die 22 is a truncated conical die plug segment 26 extending from a flat surface 23 at a first end of a cylindrical stage 28. The truncated conical die plug segment 26 tapers inwardly with increased distance from the first end of the cylindrical stage 28. The multi-layered tubing 10 is inserted into the aperture 24 until the tubing 10 makes contact with the flat surface 23 of the cylindrical stage 28. Optionally, a vacuum source 25 may be placed in communication with an opposite end of the length of tubing 10. The heating element 19 is actuated, subjecting the first die 22 to heat until the temperature of an end region of the inner layer 12 of the multi-layered tubing 10 exceeds its glass transition stage threshold, allowing deformation of that heated end region of that inner layer 12. The inserted end of the multi-layered tubing 10 is urged toward the conical die plug segment 26, preferably by mechanical means, such as a pneumatically-powered tubing grip/actuation assembly 27. The conical surface of the truncated conical die plug segment 26 extending from the flat surface 23 of the cylindrical stage 28 urges the heated region of the deformable inner layer 12 radially outwardly and/or axially. As a result, an end region 30 of the multi-layered tubing 10 is rendered thinner, and the external layer 14 along that end region 30 is received in an annular recess 32 of the first die 22 that is defined by an outer wall 31 of the cylindrical stage 28 and an inner wall 34 of the first die 22. The annular recess 32 has a thickness greater than a thickness of the external layer 14 of the multi-layered tubing 10, but less than a combined thickness of the external layer 14 and at least one inner layer 12. As used herein, it is to be understood that urging the first end of the multi-layered tubing in a direction toward a die or die segment includes actuation of the multi-layered tubing 10 while the die remains stationary, the multi-layered tubing 10 being held in place and the die urged toward the multi-layered tubing 10, and both the multi-layered tubing 10 and the die being actuated in the direction toward one other. In other words, it is the relative movement of the multi-layered tubing and the die toward one another, regardless of which may be physically stationary.

With reference to FIG. 4, the multi-layered tubing 10 is illustrated upon withdrawal from the first die station 16. It will be appreciated that along the end region 30 of the tubing 10, the core layer 12 has been pushed back away from the first end 36 of the multi-layered tubing 10, leaving only the exterior layer 14, an internal surface of which is now exposed along the end region 30 of the multi-layered tubing 10.

Turning to FIG. 5, an intermediate or second die station 18 is provided with an intermediate die, or second die 38. The second die 38 has an internal geometry including an elongate conical die plug 40. The elongate conical die plug 40 preferably has the same opening angle 0 of the truncated conical die plug segment 26, but extends a longer distance into the die 38. The vacuum source 25 may again be applied to the opposite end of the multi-layered tubing 10. The end region 30 of the tubing 10 is inserted into an aperture in the intermediate or second die 38, heat is applied to the second die 38, and the inserted end of the multi-layered tubing 10 is urged toward the elongate conical die plug 40 (or, alternatively, the multi-layered tubing 10 is fixed in place and the elongate conical die plug 40 is actuated toward the multi-layered tubing 10). Upon withdrawing the multi-layered tubing 10 from the second die 38, as illustrated in FIG. 6, it is seen that the exterior layer 14 along the end region 30 of the tubing has been further thinned out as a result of the processing at the second die station 18, into a shape complementary to the conical surface of the elongate conical die plug 40.

A third die 42 is provided at a third die station 20, and is illustrated in FIG. 7. The third die 42 is provided with a rounded (which may include, by way of example, dome-shaped or bullet-nosed) recess 44 in an aperture thereof. The vacuum source 25 may be applied to the opposite end of the multi-layered tubing 10, and the end region 30 of the tubing 10 is inserted into the third die 42. Heat is applied to the third die 42. The inserted end of the multi-layered tubing 10 is urged toward the rounded recess 44. Upon removal of the multi-layered tubing 10 from the third die 42, as illustrated in FIG. 8, it is seen that the end region 30 of the tubing 10 has been formed into a rounded shape suitable for use as the insertion end of a catheter. The rounded insertion end is free of regions prone to bald spots, because the lower melting point core layer 12, which tends to melt out and cover part of the tip of the catheter during conventional tipping processes, was urged back away from the end region 30 of the tubing 10 prior to forming the rounded insertion end at the third die station 20.

In a preferred embodiment of the present disclosure, the dies 22, 38, 42 are arranged such that the end region 30 of the tubing 10 is urged vertically upward into an aperture of the respective dies. In this manner, gravity assists in obtaining the desired flow of material of the multi-layered tubing 10 upon exposure to concentrated heat from the dies 22, 38, 42. However, it is not required to orient the dies 22, 38, 42 in such a manner. The vacuum source described above as being optionally applied to the end of the tubing 10 opposite to that inserted into the dies 22, 38, 42 is also found to assist in obtaining the desired flow of material of the multi-layered tubing 10 upon exposure to concentrated heat from the dies 22, 38, 42, but is not necessary to obtain the benefits of the present disclosure.

It is further recognized that, while the above-described method and apparatus involves the use of a series of three die stations 16, 18, 20, each with a different respective die 22, 38, 42, the benefits of the present disclosure may be obtained with as few as two different dies, including a first die with an internal geometry that, upon exposure to heat, melts back a sufficient portion of the core layer 12 of the multi-layer tubing 10 and thins out the external layer 14, and a subsequent die with a rounded recess to impart a rounded end to the thinned-out external layer 14.

While various embodiments of the present disclosure have been described herein, it will be understood that variations may be made that are still within the scope of the appended claims.

1. A method of forming a rounded catheter tip on an end of a length of multi-layered tubing comprising:
inserting a first end of the multi-layered tubing in an aperture of a first die, the first die including a cylindrical stage, a truncated conical die plug segment extending from a first end of the cylindrical stage and tapering radially inwardly with increasing distance from the first end of the cylindrical stage, and an inner wall of the first die spaced radially outwardly from the cylindrical stage, the inner wall of the first die and an outer wall of the cylindrical stage defining an annular recess therebetween having a thickness greater than a thickness of an external layer of the multi-layered tubing and less than a combined thickness of the external layer of the multi-layered tubing and at least one inner layer of the multi-layered tubing, until the inner layer of the multi-layered tubing contacts the first end of the cylindrical stage;
applying heat to the first die so that the temperature of an end region of the inner layer of the multi-layered tubing exceeds a glass transition stage threshold of the material of which the inner layer is formed, thereby rendering deformable the end region of the inner layer of the multi-layered tubing, and permitting an end region of the external layer of the multi-layered tubing to be received in the annular recess;
urging the first end of the multi-layered tubing in a direction toward the truncated conical die plug segment and the cylindrical stage of the first die;
withdrawing the multi-layered tubing from the first die;
then, inserting the first end of the multi-layered tubing in an aperture of a subsequent die, the subsequent die including a rounded recess;
applying heat to the subsequent die;
urging the first end of the multi-layered tubing in a direction toward the rounded recess, thereby modifying the end region of the external layer of the multi-layered tubing into a rounded shape complementary to a surface of the rounded recess; and
withdrawing the multi-layered tubing from the subsequent die.

2. The method of claim 1, further comprising, after withdrawing the multi-layered tubing from the first die and prior to inserting the first end of the multi-layered tubing in an aperture in the subsequent die, inserting the first end of the multi-layered tubing in an aperture of an intermediate die, the intermediate die including an elongate conical die plug;
applying heat to the intermediate die so that the temperature of the external layer of the multi-layered tubing exceeds a glass transition stage threshold of the material of which the external layer is formed, thereby rendering deformable the end region of the external layer of the multi-layered tubing;
urging the first end of the multi-layered tubing in a direction toward the elongate conical die plug, thinning out the end region of the external layer of the multi-layered tubing into a shape complementary to a conical surface of the elongate conical die plug; and
withdrawing the multi-layered tubing from the intermediate die.

3. The method of claim 2, wherein in inserting the first end of the multi-layered tubing in the aperture of the intermediate die, the elongate conical die plug has an opening angle equal to an opening angle of the truncated conical die plug segment of the first die.

4. The method of claim 2, wherein at least one of the dies is oriented vertically, with the aperture thereof arranged so as to be downwardly open, whereby upon the application of heat to the so-oriented die, flow of deformable material of which at least one layer of the multi-layered tubing is constructed is facilitated by gravity.

5. The method of claim 1, wherein at least one of the first and subsequent dies is oriented vertically, with the aperture thereof arranged so as to be downwardly open, whereby upon the application of concentrated heat to the so-oriented die, flow of deformable material of which at least one layer of the multi-layered tubing is constructed is facilitated by gravity.

6. The method of claim 1, further comprising applying a vacuum source to an end of the multi-layered tubing opposite the first end of the multi-layered tubing and activating the vacuum source while the first end of the multilayered tubing is inserted in at least one of the dies.

7. The method of claim 1, wherein in inserting the first end of the multi-layered tubing in an aperture of a first die, the inner layer of the multi-layered tubing is made of a material that is PVC-free, and the external layer of the multi-layer tubing is made of a polyether block amide thermoplastic elastomer.

8. An apparatus for providing a rounded catheter tip on a length of multi-layered tubing, comprising:
a first die station including an aperture within which is provided a first die having a cylindrical stage, a truncated conical die plug segment extending from a first end of the cylindrical stage and tapering radially inwardly with increasing distance from the first end of the stage, and an inner wall of the first die spaced radially outwardly from the cylindrical stage, the inner wall of the first die and an outer wall of the cylindrical stage defining an annular recess therebetween having a thickness greater than a thickness of an external layer of a multi-layered tubing to be inserted in the first die and less than a combined thickness of the external layer and at least one inner layer of a multi-layered tubing to be inserted in the first die; and
a subsequent die station including a subsequent die having an aperture with a rounded recess therein.

9. The apparatus of claim 8, further including an intermediate die station, the intermediate die station including an intermediate die having an elongate conical die plug in an aperture thereof.

10. The apparatus of claim 9, wherein the elongate conical die plug has an opening angle equal to an opening angle of the truncated conical die plug segment of the first die.

11. The apparatus of claim 9, wherein the subsequent die is oriented vertically, with the aperture thereof arranged so as to be downwardly open.

12. The apparatus of claim 8, wherein at least one of the first and subsequent dies is oriented vertically, with the aperture thereof arranged so as to be downwardly open.

13. The apparatus of claim 8, further comprising a vacuum source connectable to an end of a length of multi-layered tubing to be inserted into the aperture of one of the dies opposite to an end of the multi-layered tubing to be inserted into the aperture of one of the dies.

14. A method of forming a rounded catheter tip on an open end of a length of multi-layered tubing having an external layer and at least one inner layer wherein each of the external layer and inner layer has an end region adjacent the open end of the multi-layered tubing, the method comprising:
heating the end region of the inner layer of the multi-layered tubing to render the end region of the inner layer deformable;
pushing the deformable end region of the inner layer away from the open end of the multi-layered tubing and into the tubing;
heating the end region of the external layer of the multi-layered tubing to render the end region deformable; and
forming the end region of the external layer of the multi-layered tubing into a rounded tip.

15. The method of claim 14, further comprising, prior to forming the end region of the external layer of the multi-layered tubing into a rounded tip, tapering the end region of the external layer of the multi-layered tubing.

16. The method of claim 14 wherein the end region of the inner layer of the multi-layered tubing is heated to a temperature that exceeds a glass transition stage threshold of the material of which the inner layer is formed.

17. The method of claim 14 wherein the end region of the external layer of the multi-layered tubing is heated to a temperature that exceeds a glass transition stage threshold of the material of which the external layer is formed.

18. The method of claim 14, further comprising applying a vacuum source to an end of the multi-layered tubing opposite the open end of the multi-layered tubing and activating the vacuum source.

19. The method of claim 14, wherein the inner layer of the multi-layered tubing is made of a material that is PVC-free, and the external layer of the multi-layer tubing is made of a polyether block amide thermoplastic elastomer.
